(19) United States
(12) Patent Application Publication
(10) Pub. No.: US 2026/0345396 A1
(43) Pub. Date: Nov. 5, 2026

(54) VOIDING DATA LOGGING DEVICE SENSING A FILLING STATE DURING AN ACQUISITION CYCLE

(71) Applicant: Essity Hygiene and Health Aktiebolag, Goteborg (SE)

(72) Inventor: Henrik Pettersson-Falk, Göteborg (SE)

(21) Appl. No.: 19/104,982

(22) PCT Filed: Sep. 22, 2022
(86) PCT No.: PCT/EP2022/076369

§ 371 Date: Feb. 19, 2025

(51) Int. Cl. A61F 13/42; G16H 40/63

(52) U.S. Cl. CPC A61F 13/42; G16H 40/63; A61F 2013/424

(57) ABSTRACT

Disclosed is a voiding data logging device including a connector to a sensor array with a plurality of sensor areas arranged to sense a filling state of an absorbent material in the vicinity of a corresponding one of said sensor areas and a data processor configured to store acquisition information indicating at least one of said sensor areas to be read out during an acquisition cycle, read out during the acquisition cycle and via the connector at least the one sensor area indicated by the acquisition information when a sensor array is connected, to determine whether the read out sensor area indicates an above-threshold filling state of the absorbent material in the vicinity of the read out sensor area, and to change the acquisition information se to indicate that a previously read out sensor area is not read out during a subsequent acquisition cycle if when above the above-threshold filling state.

### CROSS REFERENCE TO RELATED APPLICATIONS

This application is a National Stage Application of PCT/EP2022/076369, filed Sep. 22, 2022, the contents of which are incorporated by reference in their entirety herein.

### TECHNICAL FIELD

The present invention relates to a voiding data logging device to be used with a sensor array arranged to sense a filling state of an absorbent material. The present invention further relates to the use of absorbent articles such as diapers in daycare, homecare, nursing home and hospital environments. The present invention in particular relates to improving the aspects of monitoring and managing the use of absorbent articles.

### BACKGROUND

Absorbent articles are used for addressing conditions such as leakage issues or incontinence. Such absorbent articles, mostly in the form of diapers, are likewise used for individuals but applied and managed by nursing personnel in the context of nursing homes, hospitals and home care environments. In each such environment, the filling state and the need for replacement of a full absorbent article are of considerable interest, as well as the assessment of an individual's specific voiding behavior. Especially the latter is an important input regarding the possibility of forecasting the use of absorbent articles, the planning of replacement schedules and the managing of sufficient supply in both individual environments as well as in larger contexts of the aforementioned nursing homes and the like. For example, there are techniques for detecting the filling state of absorbent articles that consider the use of conducting wires integrated into a e.g. diaper's liquid absorbent layer. A logging device is attached to these wires for sensing physical observables so as to determine the occurrence of voiding events and/or to measure some indication to the voided volume, absorbed volume, and/or absorption capacity.

Such logging devices are usually collocated with the patient and in most cases affixed to the absorbent article in the form of a diaper. Sensing a filling state of an absorbent material usually employs an interaction between a sensor and the absorbed liquid, which is predominantly water. For example, water is suitable for changing a resistance or a capacitance that can be read out by an electronic circuit. As, however, the logging devices are usually battery powered the power consumption by both measuring the base observables as well as storing and/or communicating the measured results are to be considered. Especially in the context of detecting a liquid (e.g. water) in a reliable fashion may require power resources that may be challenging for a battery powered device when also the battery life-time, and, with this, the effective up time of the logging device is taken into account.

There is therefore a need for improved voiding data logging devices that make efficient use of battery power resources while taking into account the specific environment of detecting a liquid in an absorbent material or article containing the same. Likewise, there is a need for an accordingly improved absorbent article, such as a diaper.

### SUMMARY

The mentioned problems and drawbacks are addressed by the subject matter of the independent claims. Further preferred embodiments are defined in the dependent claims. Specifically, the embodiments of the present invention may provide substantial benefits that are described in part herein.

According to one aspect of the present invention there is provided a voiding data logging device including a connector to a sensor array including a plurality of sensor areas arranged to sense a filling state of an absorbent material in the vicinity of a corresponding one of said sensor areas and a data processor configured to store acquisition information indicating at least one of said sensor areas to be read out during an acquisition cycle; read out during the acquisition cycle and via the connector at least the one sensor area indicated by the acquisition information when a sensor array is connected, to determine whether or not the read out sensor area indicates an above-threshold filling state of the absorbent material in the vicinity of the read out sensor area, and to change the acquisition information so that it indicates that a previously read out sensor area is not read out during a subsequent acquisition cycle if it was determined that it indicates the above-threshold filling state.

According to one aspect of the present invention there is provided an absorbent article including a number of sensor areas arranged to sense a filling state of an absorbent material in the vicinity of a corresponding one of said sensor areas, a connector to a voiding data logging device configured to read out via the connector at least a part of the number of sensor areas when the data logging device is connected, and an encoding circuit encoding a number and/or an arrangement in relation to the sensor areas of said sensor array.

According to one aspect of the present invention there is provided a sensor array including a number of sensor areas arranged to sense a filling state of an absorbent material in the vicinity of a corresponding one of said sensor areas, a connector to a voiding data logging device configured to read out via the connector at least a part of the number of sensor areas when the data logging device is connected, and an encoding circuit encoding a number and/or an arrangement in relation to the sensor areas of said sensor array.

### BRIEF DESCRIPTION OF THE DRAWINGS

Embodiments of the present invention, which are presented for better understanding the inventive concepts but which are not to be seen as limiting the invention, will now be described with reference to the figures in which:

FIGS. 1A and 1B show schematic views of applications of respective voiding data logging devices according to embodiments of the present invention;

FIG. 2 shows a schematic view of a voiding data logging devices according to embodiments of the present invention;

FIGS. 3A and 3B show schematic views of an operation sensor of voiding data logging devices according to embodiments of the present invention;

FIGS. 4A to 4D show schematic views of encoding different types in the context of embodiments of the present invention.

FIGS. 5A to 5C show schematic views of different arrangements within a sensor arrangement according to embodiments of the present invention.

### DETAILED DESCRIPTION

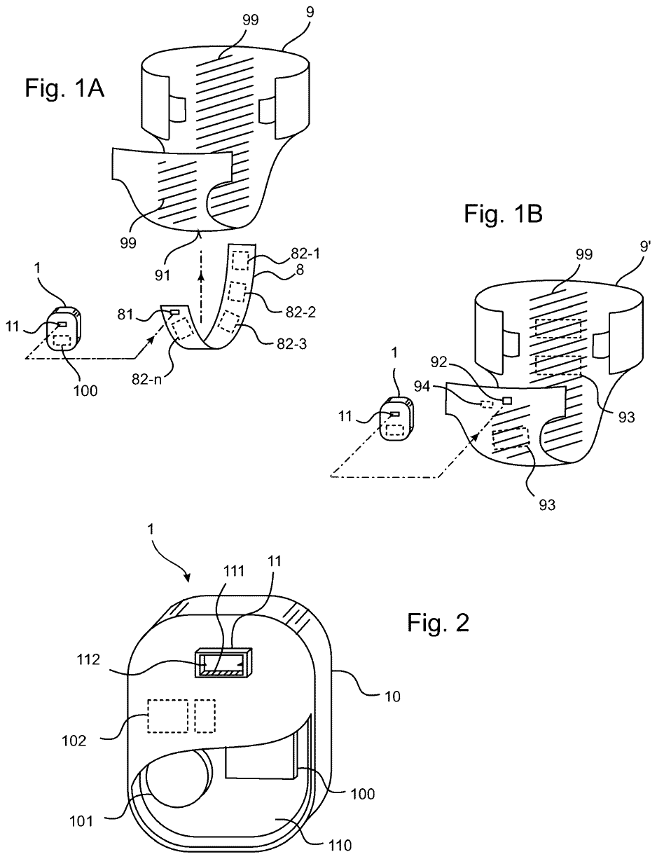

FIGS. 1A and 1B show schematic views of applications of respective voiding data logging devices according to embodiments of the present invention. FIG. 1A focusses on embodiments in which the sensor array is as such independent from an absorbent article. Namely, this figure shows as an example for this situation the absorbent article as a diaper 9 that has an absorbent material 99 in the form of a liquid absorbent layer located across the applicable and as such usual areas of the diaper 9. The sensor array is shown as a strip-like sensor arrangement 8, which can be affixed to an outer surface 91 of the diaper 9 by means of, for example, a part of a hook-and-loop-fastener, an adhesive, etc. Likewise, any other suitable means, for example including the option to have some kind of pocket, pouch, hooks or strips at the diaper 9 so as to keep the sensor arrangement 8 at a sufficiently constant position relative to the diaper during use (i.e. during a time the diaper is worn by a user or patient and/or the logging of voiding shall take place).

A voiding data logging device 1 is provided including a connector 11 which can establish the desired electric contacts to the sensor array as part of the strip-like sensor arrangement 8. For this purpose, the strip-like sensor arrangement 8 may include a corresponding connector 81 that mates with the connector 11 of the voiding data logging device 1 and which may arranged to establish not only a reliable electric connection for the applicable data-, measurement-, and supply-lines, but also a mechanical engagement so as to hold the voiding data logging device 1 reliably at the strip-like sensor arrangement 8 during a time when the user/patient wears the diaper 9.

In general, the sensor array, here shown as a part of the strip-like sensor arrangement 8, includes a plurality of sensor areas that arranged to sense a filling state of an absorbent material in the vicinity of a corresponding one of said sensor areas. The sensor array can thus be a sensor strip with the plurality of sensor areas arranged along a line in a main extension direction of the sensor strip. For example, the sensor areas may include electrodes, coplanar capacitors, inductive coils, resistors, and the like for establishing a predetermined relation between the filling state of the absorbent material in the corresponding vicinity of a sensor area and the electrical observable that may depend on the chosen type of the sensor areas (i.e. capacitance, inductivity, resistance, etc.).

The voiding data logging device 1 further includes a data processor 100 that is configured to store acquisition information indicating at least one of said sensor areas to be read out during an acquisition cycle. Acquisition information may generally refer to a collection of information that determines whether, when and/or how a signal is acquired from a sensor array. Namely, a signal may be acquired in an acquisition sequence during which a test signal is applied to a sensor and the response is measured. For example, an ac signal can be implied for measuring the impedance response by the sensor. Likewise, a voltage may be applied so as to measure a current, and, with this, an effective resistance of the sensor. This measurement of the desired observable may thus take place at specific timings whereas substantially no acquisition takes place in the time spans between two subsequent acquisition sequences. In case that there is no particular sequence of actions concerned for acquiring the sensor data one may also refer to an instance of reading out the corresponding sensor area. In any way, however, one such time span during which at least one acquisition sequence is performed is referred to as an acquisition cycle. Further, the voiding data logging device 1 may consume a relative high power from a power source during acquisition sequences, while it may consume a relative low power from the power source during times between acquisition sequences.

The data processor 100 that also configured to read out during the acquisition cycle, e.g. during a corresponding acquisition sequence or instance, and via the connector 11 at least the one sensor area indicated by the acquisition information when a sensor array is connected. Specifically, the acquisition information may define what sensor area of the plurality of connected sensor areas is to be read out and indicate when such reading out shall take place. In this way, the acquisition information can define an acquisition pattern maps to a linear arrangement of sensor areas in a connected sensor array, and the data processor stores an initial acquisition pattern considering a center sensor area for reading out. For example, the definition of what sensor area is to be read out may include information related to a pointer or and identifier that can be used to select one particular sensor area from said plurality. Such pointer may be in the form of a number that addresses the sensor area in a linear or elsehow suitable arrangement and addressing scheme. The indication on a time may for example include information on a length of an acquisition cycle, so that data processor 100 can wait for that time to elapse for initiating an acquisition sequence or instance.

The data processor 100 is also configured to determine whether or not the read out sensor area indicates an above-threshold filling state of the absorbent material in the vicinity of the read out sensor area. Specifically, the data processor may store some information on a threshold that can be used to distinguish a filling state from an empty state, in which the latter characterized a state in which the absorbent material has essentially not yet absorbed any liquid, such as a bodily fluid in the context of the application as voiding data logging. Such an empty state is to be considered as a state in which the absorbent material in one area of the absorbent article has not been used so far and is accordingly able to absorb a liquid during any further voiding event(s). The presence of at least one empty or non-filled state area may indicate that the absorbent article can still remain in use and does not yet need to be exchanged. Further, it may take several sequences or instances of reading out one particular sensor area to ascertain that an above-threshold filling state is reached. For example and in order to achieve increased reliability, a number of consecutive read-outs with (in tolerance limits) stable values may be needed before it is determined an above-threshold filling state of the absorbent material in the vicinity of the read out sensor area.

The data processor 100 is further configured to change the acquisition information so that it indicates that a previously read out sensor area is not read out during a subsequent acquisition cycle if it was determined that it indicates the above-threshold filling state. Specifically, the acquisition information can be changed that a hitherto addressed or indicated sensor area is at least not read out during a subsequent acquisition cycle when another sensor area should be read out. In this way, energy resources are saved in the voiding data logging device in that no power is consumed for reading out a sensor area that was recently determined to indicate above-threshold filling state and is thus—at least for the time being—of lesser interest. Likewise, the acquisition information can be changed that a hitherto addressed or indicated sensor area is not again read out during—possibly any—later acquisition cycle. In this way, the data processor changes the acquisition information so that it indicates that a next sensor area different from said previously read out sensor area is read out during a subsequent acquisition cycle. More specifically, the data processor can change the acquisition information so that it indicates a next sensor area neighboring the previously read out sensor area. In general, however, a hitherto addressed or indicated sensor area can still be read out later, but with a lower frequency, even if it was previously determined to indicate above-threshold filling state for the purpose of, for example, verification and/or confirmation purposes.

For example, the acquisition information can be changed to define that another sensor area of the plurality of connected sensor areas is to be read out and/or that information related to a pointer or and identifier select one particular other sensor area from said plurality. In case such a pointer is in the form of a number that addresses the sensor area in a linear or elsehow suitable arrangement and addressing scheme, that pointer may be simply incremented or decremented, depending on the addressing direction. The acquisition information may still indicate when a corresponding next reading out shall take place. The indication on a time may for example again include information on a length of an acquisition cycle, so that data processor 100 can wait for that time to elapse for initiating an acquisition sequence or instance. Said time, however, can also change, as embodiments envisage situations in which the acquisition cycle may change with one or more sensor areas having found to be in an above-threshold filling state. For example, the cycle may be decreased so as to obtain a finer time resolution toward the end of an absorbent article's lifetime and, with this, being able to appropriately determine that an absorbent article may need to be replaced.

In some embodiments the data processor can be configured to read out one of the plurality of sensor areas of a connected sensor array by applying some sort of stimulator signal a sensing respective response. For example, the data processor may include a network analyzing function and is configured to read out a sensor area of a connected sensor array by applying an ac signal, detecting a response and determining an indicator on an impedance of the sensor area. Specifically, the data processor 100 may include a network analyzing functionality that applies an AC signal to capacitor electrodes of a sensor array and determines the reflected and/or absorbed power, which will change due to a change in effective impedance of the sensor area (in the form of a capacitor) in response to the dielectric properties change for the capacitor as the absorbent material absorbs a liquid (e.g. bodily fluid).

In some embodiments of the voiding data logging device 1, the acquisition information further includes any one of an acquisition interval, an acquisition order and a sampling rate. Generally, the data processor can store the acquisition information in the form of an acquisition pattern determining which one or more sensor area/areas of a connected sensor array is/are read out in a subsequent acquisition cycle. Further, the data processor is configured to change a time period between two acquisition cycles if the read out sensor area indicates the above-threshold filling state.

For example, an acquisition interval may be defined by means of information that indicates a time between two subsequent acquisition sequences or instances in the form of the already discusses acquisition cycle. Said information may directly code a time, e.g. 1 or 60 for defining 1 minute and, respectively 1 hour, or indirectly code a time, e.g. 1 meaning every minute, 2 meaning every hour, 3 every second hour, and so forth. Generally, said acquisition interval may relate to how often an acquisition sequence takes place. A first acquisition interval may define roughly every minute, which is relatively often, while a second acquisition interval may define roughly every hour, which is relatively rare.

An acquisition order may be defined by means of information that indicates what sensor area of the plurality of sensor areas should be addressed. In this way, the acquisition order may indicate that a specific one of the plurality of sensor areas is to be read out during an upcoming acquisition sequence/instance or within the next acquisition cycle. The acquisition order may well indicate what specific one of the plurality of sensor areas is to be read out during any following acquisition. In this way, it can be specified what sensor area is read out at what time or under what conditions. The acquisition information may indicate a position in such an order, so that it can be simply followed a given order that may not follow a strictly linear or monotonous manner.

A sampling rate may be defined by means of information that indicates how many individual data acquisitions are made for a single sensor area during an acquisition sequence. Usually, a data acquisition may at least at some point involve an analog-to-digital conversion (ADC) during which an analog level (e.g. representing a voltage, current, resistance, or reflected power as part of a network analysis) is converted into a digital representation. During such conversion, an analog comparison level is generated and the analog input level is compared to that. This process, also referred to as sampling or supersampling, may involve a successive approximation and is thus an iterative process requiring energy and power resources. For example, 1 or more than 1, for instance up to 100, 50, 30, or 20 individual data acquisitions for a single acquisition sequence and a single sensor area may be averaged into a single acquisition data, optionally after removing non-plausible data points and/or applying other as such known data filtering techniques. A higher super sampling rate may provide a higher accuracy, however, will consume more energy.

Generally, any one or two of the acquisition interval, the acquisition order and the sampling rate may be the same, while the remaining one is changed. In some embodiments, the acquisition interval may be the same, but the degree of sampling, i.e. the number of measurements that are averaged into one measurement value may be different. For example, the interval may be 1 second, but the degree of sampling can be from 1 to 16 measurements. The higher degree of sampling results in higher battery consumption, but gives more reliable measurements. Thus, the data processor 100 is configured to adjust the acquisition scheme based on the determined array type and, in this way, it may adjust the acquisition interval and/or the acquisition order and/or the sampling rate. For example, there may be initial default values for the parameters of the acquisition interval and/or the sampling rate and once a specific number of sensor areas have been determined to indicate an above-threshold filling state, at least one of the parameters may be adapted to further reduce energy consumption in the voiding data logging device by, for example, increasing the acquisition interval (i.e. increasing the time between two subsequent acquisition cycles) and/or reducing the sampling rate.

For example, the strip-like sensor arrangement 8 may include the plurality of sensor areas 82-1, 82-2, . . . 82-n. These sensor areas can thus be located at different and specific positions relative to the absorbent article. i.e. the diaper 9. For example, the strip-like configuration of the sensor arrangement may result in that some sensor area(s) are closer to a source of voiding (e.g. close to the center of diaper 8 at a site of an ureter opening), while that some other sensor area(s) are farther away from that voiding source. It may thus be of interest to consider the sensor area(s) close to the voiding source toward the beginning of a use of a diaper, while other sensor area(s) may be of more interest toward the end of the life-time of a diaper, i.e. at times when the absorption reaches a maximum or desired target absorption capacity of the absorbent material of the diaper 9.

FIG. 1B focusses on embodiments in which the sensor array is integrated into or forms part of an absorbent article. Namely, this figure shows, as an example for this situation, the absorbent article as a diaper 9 that has again an absorbent material 99 in the form of a liquid absorbent layer located across the applicable and as such usual areas of the diaper 9. In these embodiments, the sensor array 93 is integrated into the absorbent layer or is located on the absorbent layer (the types of sensors again apply to this embodiment as they have been described elsewhere in the present disclosure). Further, the absorbent article may include connector 92 that engages with the connector 11 of the voiding data logging device 1 just in the same or in a similar way as does connector 81 that was described in conjunction with FIG. 1A. In some embodiments the absorbent article 9 includes an encoding circuit 94 encoding a number and/or an arrangement in relation to the sensor areas of said sensor array, which is described in greater details elsewhere in the present disclosure.

FIG. 2 shows a schematic views of voiding data logging devices according to embodiments of the present invention. Accordingly, the voiding data logging device 1 includes a connector 11 to a sensor array including a plurality of sensor areas arranged to sense a filling state of an absorbent material in the vicinity of a corresponding one of said sensor areas. The connector 11 is preferably arranged to establish not only a reliable electric connection for the applicable data-, measurement-, and supply-lines, but also a mechanical engagement so as to hold the voiding data logging device 1 reliably at the strip-like sensor arrangement 8 during use. For this purpose, the connector 11 may include one or more electrical contacts 111, for example in the form of some resilient contact tongue or a contact strip. The surface of such tongues and strips may be coated, for example with gold so as to ensure good contact also in wet or humid environments. The connector 11 may further include one or more mechanical elements 112, such as indents or springs which will then hold the connector 11, and, with this, the voiding data logging device 1 sufficiently secure at sensor array, sensor arrangement or strip.

The voiding data logging device 1 further includes a data processor 100 configured to read out via the connector 11 at least a part of the plurality of sensor areas when a sensor array is connected. Generally, the data processor 100 is configured to store acquisition information indicating at least one of said sensor areas to be read out during an acquisition cycle; read out during the acquisition cycle and via the connector at least the one sensor area indicated by the acquisition information when a sensor array is connected, to determine whether or not the read out sensor area indicates an above-threshold filling state of the absorbent material in the vicinity of the read out sensor area, and to change the acquisition information so that it indicates that a previously read out sensor area is not read out during a subsequent acquisition cycle if it was determined that it indicates the above-threshold filling state.

Generally, the voiding data logging device 1 may include a power source 101, e.g. a battery or high-capacity/ultra-capacitor, further elements 102 including optional memories and/or communication modules (e.g. for communication via Bluetooth™, BLE, WiFi, GPRS, GSM, PCM, UMTS, LTE, 2G, 3G, 4G, 5G and related or accordingly evolving standards). All or some of the electronic elements may be mounted on a printed circuit boards (PCB) 110 that is accommodated by a logging device housing 10. The latter may be sealed against air, liquids, humidity, dust, and the like so as to provide reliable operation in the intended environments.

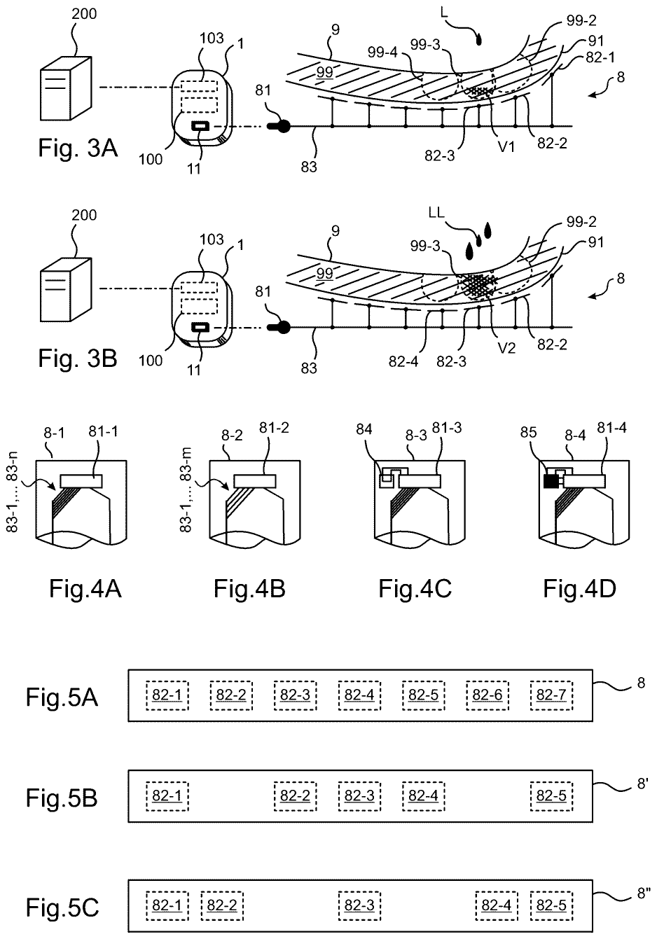

FIGS. 3A and 3B show schematic views of an operation sensor of voiding data logging devices according to embodiments of the present invention. In FIG. 3A there is shown an absorbent article in the form of a cut view of a diaper 9 including absorbent material 99 in a layer (absorbent layer) at least in some area of the diaper. A continuous layer may also be replaced by some kind of pocket configuration in which liquid flow and exchange between two pockets is substantially suppressed. An array arrangement 8 is schematically shown as to be in the vicinity of an outer surface 91 of the diaper 9. The array arrangement 8 includes a plurality of sensor areas 82-1, 82-2, . . . that are, for example, coplanar capacitors that can sense a liquid accumulation in a respective area of the absorbent material 99. Such areas are shown by the dashed lines 99-2, 99-3, . . . but are understood not to represent strictly delimited corresponding areas, as the latter may overlap at least to some extent.

An arrangement of signal lines 83 connects the sensor areas 82-1, 82-2, . . . via a connector 11/81 to a voiding data logging device 1 including at least the data processor 100 and which has been described already in conjunction with the FIGS. 1A, 1B, and 2. In this example, there is shown—albeit optional—a communication 103 that is configured to convey any relevant and desired data from the result of acquiring data from the sensor areas toward a data processing entity 200, in the exemplary form of a network server or functionality. This entity 200 may receive and further process the acquired data so as to provide logs, reports, overviews, statistical analyses, or the function of sending alerts that indicate, amongst others, a need for a replacement of the absorbent article.

In this example, the data processor 100 include a network analyzing functionality as representing the general functionalities involved with reading out a sensor area. Such a network analyzing functionality may apply an AC stimulus signal to capacitor electrodes of a sensor area and determines the reflected and/or absorbed power, which will change due to a change in effective impedance of the sensor area (in the form of a capacitor) in response to the dielectric properties change for the capacitor as the absorbent material absorbs a liquid (e.g. bodily fluid). Namely, the data processor 100 may selectively send an AC stimulus to the sensor area 82-2 that is indicated by the acquisition information. In this context, the latter may also include parameters for a specific readout, including e.g. an ID, a stimulus power, a reflection power threshold which may discriminate directly or indirectly the above-threshold filling state, and the like.

In the shown example, the absorbent material 99 in the corresponding area 99-3 has already absorbed some liquid L in a volume V1. The measured response may thus yield a result which is not an above-threshold filling state. However, this measurement during the respective acquisition sequence has already consumed power as, amongst others, some of the stimulus power is absorbed by the effective circuitry formed by the sensor in the form of a capacitor and the target in the form of liquid absorbed in the absorbent material forming that capacitor's dielectric.

FIG. 3A shows the situation the absorbent material 99 in the corresponding area 99-3 has now already absorbed some more liquid LL in a volume V2. The measured response may thus yield a result which is an above-threshold filling state, indicating that the area 99-3 can no longer absorb liquid or is due to do so. Also this measurement during the respective acquisition sequence has again consumed power as even a greater of the stimulus power is absorbed by the effective circuitry 82-3, 99-3. This consumed power may be larger than a power required for acquiring an empty sensor area or an area that is in a state below the filling state. The data processor 100 thus has determined that the read out sensor area 82-3 indicates an above-threshold filling state of the absorbent material 99 in the vicinity 99-3 of the read out sensor area 82-2. Accordingly, the data processor 100 thus changes the acquisition information so that it indicates that the previously read out sensor area 82-3 is no longer read out during a subsequent acquisition cycle. In this way, power consumption can be avoided for information that is now present in the device and no further specification is required.

Instead, the data processor 100 may change the acquisition information so that it indicates that a not yet read out sensor area is read out during a subsequent acquisition cycle. For example, the order may proceed to one or more adjacent sensor areas 82-2, 82-4 which can be assumed to be empty or yet only filled to a lower degree, which may require substantially less power from the power resources available in the voiding data logging device 1. In this way, areas are only acquired when they are empty or being filled in the sense that they already have absorbed some liquid but less than indicated by a threshold filling state. Once that has happened, i.e. a sensor area has been determined to indicate an above-threshold filling state the acquisition sequence switches to another, likely still empty, area so as to save power involved.

FIGS. 4A to 4D now show schematic views of schemes for encoding different types in the context of embodiments of the present invention. Specifically, there in such embodiments, the voiding data logging device is configured to consider different types of sensor arrangements that include different configurations (number, size, etc.) of sensor areas. Specifically, this may cooperate with further embodiments that define different layouts as considering different targets during operation, use and assessment and they are described in greater detail below in conjunction with FIG. 5A onwards.

In a first scheme, strip-like sensor arrangements 8-1 (FIG. 4A) and 8-2 (FIG. 4B) include sensor arrays of the respective type but—in manufacturing tolerances—identical connectors 81-1, 81-2. However, in one case a first, relatively high number of signal lines 83-1, . . . 83-n may be connected to connector 81 (FIG. 4A), while in another case a second, relatively low number of signal lines 83-1, . . . 83-m may be connected (FIG. 4B). In this way the processor may determine that some signal lines are not connected as the application of a test signal (e.g. test voltage or AC signal) does not yield a sensible response (e.g. open circuit or full power reflectance).

In general, the data processor 100 can be configured to read out one of the plurality of sensor areas of a connected sensor array and to determine whether or not the sensor area is present based on a sensing response. In this way, the processor can determine the type of the connected sensor array and adjust the acquisition scheme based on the determined array type. This may be a specific form voiding data logging device in which the data processor is configured to determine a number of sensor areas of a connected sensor array and to adjust the acquisition scheme based on the determined number as an array type. This may involve determining whether or not a sensor area is present on a connected sensor array. Subsequently, the data processor can then proceed by reading out via the connector a relatively large number of sensor areas for a determined first type (e.g. that of FIG. 4A) and a relatively small number of sensor areas for a determined second type (e.g. that of FIG. 4B).

FIG. 4C shows a schematic view of an embodiment considering an encoding circuit co-implemented together with the sensor array. This is an embodiment for a voiding data logging device in which the data processor is configured to read out an encoding circuit for determining the type and/or a number of connected sensor areas of a connected sensor array. The data processor may further be configured to read out such an encoding circuit for determining an arrangement of sensor areas of a connected sensor array, including information on any of a position of a senor area, a size of a sensor area, a connection scheme of two or more sensor areas (e.g. serial or parallel connection), and the like. For example and as shown, a strip-like sensor arrangement 8-3 may again include a connector 81 of which one or more signal lines lead to an encoding circuit 84. The latter can be relatively simple by differentiating between a short circuit and an open circuit between at least two signal lines toward the connector 81, or also relatively complex by including a number of such open/closed circuits to encode a binary value or even some (discrete) electronic component, such as passives incl. resistors, capacitors, inductors, and the like.

FIG. 4D shows a schematic view of an embodiment considering an encoding circuit co-implemented together with the sensor array. This is another embodiment for a voiding data logging device in which the data processor is configured to read out an encoding circuit for determining the type of a connected sensor array. The strip-like sensor arrangement 8-4 again includes a connector 81 of which one or more signal lines lead to an encoding circuit 84-2 which may be or include a relatively complex by including an active or integrated component, such as a memory, a read only memory (ROM), a serial ROM, a FLASH memory, an I2C-memory, an SPI-memory and the like. The data processor can thus read out via the connector also any stored information for determining the type of a connected sensor array. For example, the information may represent a type identifier, a serial number and/or even information on the acquisition scheme to be adopted as such. In a further embodiment the encoding circuit can provide for sending information on the type as payload together with the sensor read out to the processor.

In a further embodiment of the voiding data logging device of the present invention, the data processor may be configured to determine a parameter of a connected sensor array and to adjust the acquisition scheme based on the determined parameter. The sensor number of sensor areas may then not necessarily differ between different types. Specifically, they can be identical, except for a parameter in the possible form of a “strip type identifier” read out by the data processor. This type identifier can act as a hardware key to activate different settings regarding the acquisition interval, and optionally also different settings in the back-end and/or presentation modes in the user interface, i.e. the acquisition scheme.

FIGS. 5A to 5C show schematic views of different arrangements within a sensor arrangement according to embodiments of the present invention. Specifically, the voiding data logging device may be configured to consider different types of sensor arrangements that include different configurations (number, size, etc.) of sensor areas and define different layouts as considering different targets during operation, use and assessment. In a first exemplary option as shown in FIG. 5A, a strip-like sensor arrangement 8 includes a plurality of sensor areas 82-1, 82-2, . . . along a main longitudinal direction of the strip with a substantially equidistant interval of sensor areas. This may represent a type for universal applications as a more or less constant distance between neighbouring sensor areas may be suitable for most applications. This type, i.e. information on that equidistant spacing and optionally also the number of sensor areas may be conveyed to the voiding data logging device as described in conjunctions with FIGS. 4A through 4D.

In a further exemplary option as shown in FIG. 5B, a strip-like sensor arrangement 8′ includes again a plurality of sensor areas 82-1, 82-2, . . . along a main longitudinal direction of the strip but not with equidistant spacing of sensor areas. In particular, the shown configuration represents embodiments in which the spacing of sensor areas is smaller in the center of the strip as compared to at least one end of the strip. This may represent a type for applications in which the initial voiding behaviour is of main interest, for example as part of an assessment of a new user or patient. This arrangement may provide information on a typical location of an origin of bodily liquids so as for example the approximate position of an ureter opening. This may contribute in finding a most appropriate configuration, type or size of an absorbent article for that particular user or patient. This type may again be conveyed to the voiding data logging device as described earlier.

In a yet further exemplary option as shown in FIG. 5C a strip-like sensor arrangement 8″ includes again a plurality of sensor areas 82-1, 82-2, . . . along a main longitudinal direction of the strip but not with equidistant spacing of sensor areas. In particular, the shown configuration represents embodiments in which the spacing of sensor areas is smaller toward at least one end of the strip. This may represent a type for applications in which the initial voiding behaviour is of subordinate interest, and the main focus resides withing determining an appropriate moment for changing an absorbent article as it reaches an overall maximum capacity of absorption. In this way, the available power resources are reserved for the acquisition of data during the time of highest interest, i.e. the time when a diaper runs full. This type may again be conveyed to the voiding data logging device as described earlier.

Although detailed embodiments have been described, these only serve to provide a better understanding of the invention defined by the independent claims and are not to be seen as limiting.

1. A voiding data logging device comprising a connector to a sensor array comprising a plurality of sensor areas arranged to sense a filling state of an absorbent material in the vicinity of a corresponding one of said sensor areas and a data processor configured to:
store acquisition information indicating at least one of said sensor areas to be read out during an acquisition cycle;
read out during the acquisition cycle and via the connector at least the one sensor area indicated by the acquisition information when a sensor array is connected,
to determine whether or not the read out sensor area indicates an above-threshold filling state of the absorbent material in the vicinity of the read out sensor area, and to
change the acquisition information so that it indicates that a previously read out sensor area is not read out during a subsequent acquisition cycle if it was determined that it indicates the above-threshold filling state.

2. The voiding data logging device according to claim 1, wherein the data processor changes the acquisition information so that it indicates that a next sensor area different from said previously read out sensor area is read out during a subsequent acquisition cycle.

3. The voiding data logging device according to claim 2, wherein the data processor changes the acquisition information so that it indicates a next sensor area neighboring the previously read out sensor area.

4. The voiding data logging device according to claim 1, wherein the data processor stores the acquisition information in the form of an acquisition pattern determining which one or more sensor area/areas of a connected sensor array is/are read out in a subsequent and/or later acquisition cycle.

5. The voiding data logging device according to claim 4, wherein the acquisition pattern maps to a linear arrangement of sensor areas in a connected sensor array, and the data processor stores an initial acquisition pattern considering a center sensor area for reading out.

6. The voiding data logging device according to claim 1, wherein the data processor is configured to read out an encoding circuit for determining a number of sensor areas of a connected sensor array.

7. The voiding data logging device according to claim 1, wherein the data processor is configured to read out an encoding circuit for determining an arrangement of sensor areas of a connected sensor array.

8. The voiding data logging device according to claim 1, wherein the data processor comprises a network analyzing function and is configured to read out a sensor area of a connected sensor array by applying an ac signal, detecting a response and determining an indicator on an impedance of the sensor area.

9. The voiding data logging device according to claim 1, wherein the data processor is configured to change a time period between two acquisition cycles if the read out sensor area indicates the above-threshold filling state.

10. The voiding data logging device according to claim 9, wherein the data processor is configured to reduce a time period between two acquisition cycles if the read out sensor area indicates the above-threshold filling state.

11. An absorbent article comprising a number of sensor areas arranged to sense a filling state of an absorbent material in the vicinity of a corresponding one of said sensor areas, a connector to a voiding data logging device configured to read out via the connector at least a part of the number of sensor areas when the data logging device is connected, and an encoding circuit encoding a number and/or an arrangement in relation to the sensor areas of said sensor array.

12. A sensor array comprising a number of sensor areas arranged to sense a filling state of an absorbent material in the vicinity of a corresponding one of said sensor areas, a connector to a voiding data logging device configured to read out via the connector at least a part of the number of sensor areas when the data logging device is connected, and an encoding circuit encoding a number and/or an arrangement in relation to the sensor areas of said sensor array.
